(19) United States
(12) Patent Application Publication
(10) Pub. No.: US 2015/0038890 A1
(43) Pub. Date: Feb. 5, 2015

(54) Knee Immobilizer Support Cuff

(71) Applicants: Clinton James Montgomery, Las Vegas, NV (US); Elicia Dene' Montgomery, Las Vegas, NV (US)

(72) Inventors: Clinton James Montgomery, Las Vegas, NV (US); Elicia Dene' Montgomery, Las Vegas, NV (US)

(21) Appl. No.: 13/955,250

(22) Filed: Jul. 31, 2013

(51) Int. Cl. A61F 5/01

(52) U.S. Cl. CPC A61F 5/0123
USPC 602/26

(57) ABSTRACT

The Knee Immobilizer Support Cuff has been created as a medical apparatus for bottom based support for a knee immobilizer (brace). Knee Immobilizers are difficult to keep in the correct position and often slip down the patient's leg during upright orientation (standing/walking). The Knee Immobilizer Support Cuff will stabilize the knee immobilizer without having to overly tighten the straps, thereby avoiding the discomfort and restriction of blood flow to the knee and surrounding tissues. The knee immobilizer support cuff will be offered in a variety of sizes and/or shapes to fit the needs of the individual patient.

### CROSS-REFERENCE TO RELATED APPLICATIONS

None

### FEDERALLY SPONSORED RESEARCH OR DEVELOPMENT

No federal funding has been used or will be used in the development of this product.

### BACKGROUND OF THE KNEE IMMOBILIZER SUPPORT CUFF

The Knee Immobilizer Support Cuff has been created for application in the medical field as a support apparatus to aid in the stabilization of any prescribed Knee Immobilizer. Knee Immobilizers are common appliances prescribed by a medical doctor (usually an Orthopedic Surgeon) to stabilize the knee joint after injury or surgical procedure. These immobilizers are difficult to keep in the correct position and often slip down the patient's leg during upright orientation (standing/walking). Currently, the only recommendation from many Orthopedic Surgeons and Physical Therapists is to tighten the straps on the knee immobilizer. This solution results in two negative consequences; discomfort, especially after surgery; and a restriction of blood flow to the knee and surrounding tissues.

### SUMMARY

To remedy the problem noted above, the Knee Immobilizer Support Cuff provides continuous support below the knee immobilizer. The cuff, which is designed to fit around the bottom of the leg resting on the ankle, provides an impediment to the forces of gravity which push the immobilizer down. The Knee Immobilizer Support Cuff is to be offered in several sizes, all of which are adjustable to fit the individual patient.

### DRAWINGS/IMAGES

FIG. 1: Graphic of a leg with an unsupported knee immobilizer demonstrating the problem with slipping.

FIG. 2: Graphic of a leg with the Knee Immobilizer Support Cuff supporting the knee immobilizer from the ankle.

FIG. 3: Graphic of the Side and Top View of the Knee Immobilizer Support Cuff the height and diameter will vary based on the size required by the patient.

FIG. 4: Photograph of an unsupported knee immobilizer.

FIG. 5: Photograph of a prototype Knee Immobilizer Support Cuff supporting a knee immobilizer from the ankle above the shoe.

### DESCRIPTION

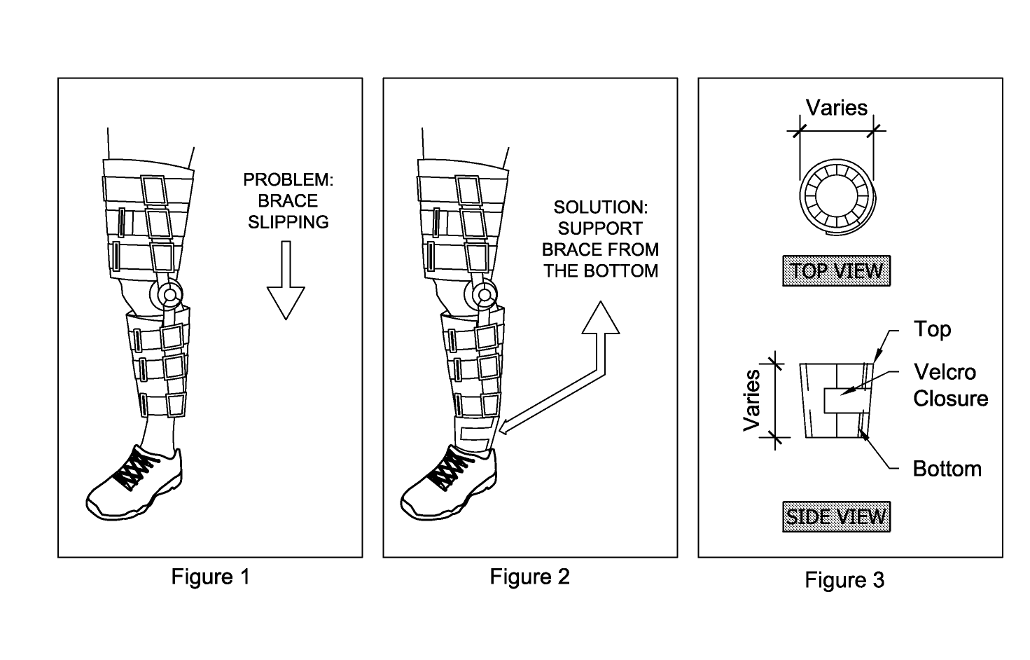

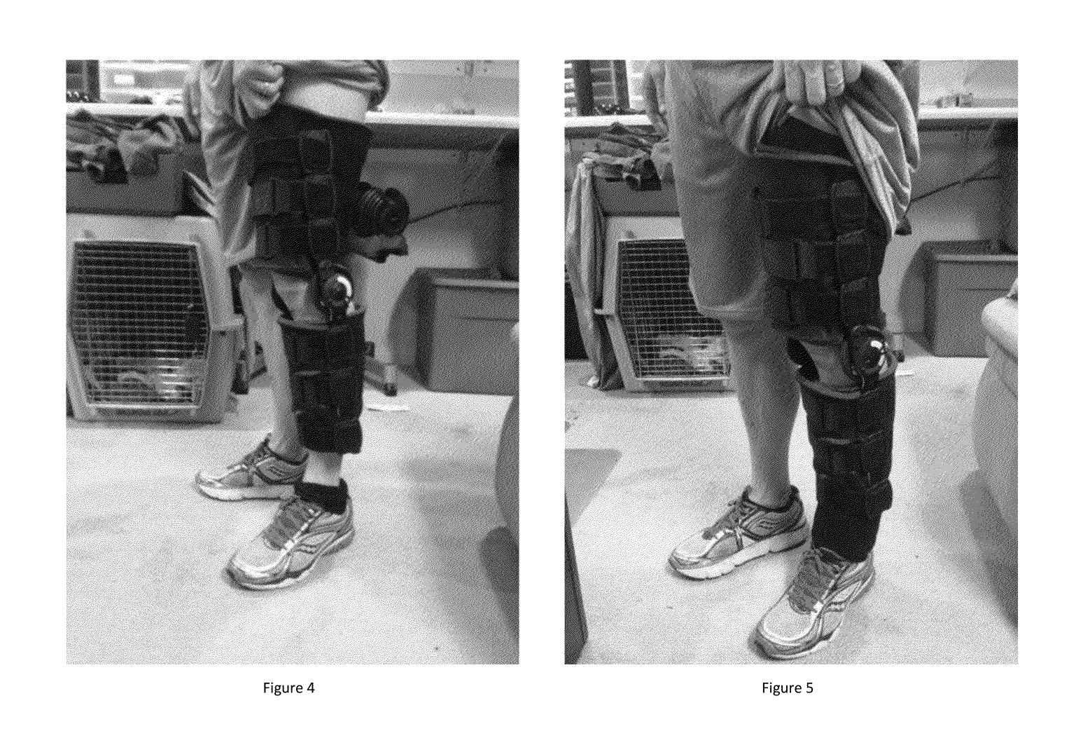

This device's intention is to prevent a knee immobilizer apparatus from sliding down the leg due to gravity and movement (FIG. 1). Traditional theory suggests to simply tighten the knee immobilizer so that it won't slide down as easy. My idea is such that if a cuff is used from underneath, the knee immobilizer will not slide down the leg as it is prevented from doing so (FIG. 2). It is not really the weight of the knee immobilizer, but rather gravity and the anatomy of a typical leg that causes the knee immobilizer to wiggle down the leg and out of position. I have developed and implemented a prototype that works well. I have had a recent ACL surgical repair to my left knee which currently requires the use of a full leg knee immobilizer. The knee immobilizer stays in place nicely with the use of my prototype.

The prototype is made of a soft jersey knit fabric and is formed with two layers of half inch foam. It is approximately five (5) inches high and twelve (12) inches long when flattened out. It wraps around the lower leg and secures with a Velcro tab.

The finished product will be less bulky than the one shown in FIG. 5 with a size diameter and height to vary to fit the individual patient or generically as Small, Medium, Large and X-Large. The finished shape of the cuff may vary slightly from the one shown in FIG. 5.

The embodiment of the invention in which an exclusive property or privilege is claimed are defined as follows:

1. A medical appliance cuff device that is worn at or above the ankle and is intended to support any knee immobilizer by creating a barrier above the ankle to prevent the immobilizer from slipping down the leg.
